(19) United States
(12) Patent Application Publication
(10) Pub. No.: US 2005/0054838 A1
(43) Pub. Date: Mar. 10, 2005

(54) Method of isolating and purifying aequorin, aequorin produced by the method, and process for detecting calcium ions with aequorin

(75) Inventors: Motohiro Otsuka, Tokyo (JP); Hiroshi Mizuno, Tsukuba-shi (JP); Frederic Ichiro Tsuji, San Diego, CA (US); Hiromi Takenaka, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: NEC SOFT, LTD.; National Institute of Agrobiological Sciences

(21) Appl. No.: 10/895,900

(22) Filed: Jul. 22, 2004

(30) Foreign Application Priority Data
Aug. 29, 2003 (JP) 305743/2003

(51) Int. Cl. C12Q 1/68; A23J 1/00; C07K 1/00; C07K 14/00; C07K 16/00; C07K 17/00

(52) U.S. Cl. 530/412

(57) ABSTRACT

Isoforms of apoaequorin and isoforms of aequorin are isolated and purified from recombinant apoaequorin and a solution containing regenerated aequorin, respectively, by gradient elution chromatography. As a result, aequorin can be isolated and purified.

This application claims priority to Japanese Patent Application JP2003-305743, the disclosure of which is incorporated herein by reference.

### BACKGROUND OF THE INVENTION

1. Field of the Invention

The present invention relates to methods of regenerating, isolating, and purifying aequorin obtained from a culture filtrate containing apoaequorin extracellularly produced by deoxyribonucleic acid (DNA) recombination techniques, wherein the apoaequorin is a protein moiety of aequorin, a calcium-sensitive luminescent protein.

2. Description of the Related Art

Aequorin is a luminescent material isolated from Aequorea victoria and is a complex of apoaequorin, coelenterazine, and molecular oxygen. Aequorea victoria is a bioluminescent creature and has photocytes which can emit green light in the margin of its umbrella. The luminescence is due to the presence of two proteins, namely aequorin and a green fluorescent protein (hereinafter, referred to as “GFP”). The aequorin is a photoprotein that can bind to divalent calcium ions (Ca²⁺).

Apoaequorin is a protein composed of 189 amino acid residues and has four EF-hand structures that can bind to three calcium ions and has three cysteine residues. Apoaequorin has a molecular weight of 21.4 kDa.

Binding of calcium ions to aequorin readily causes an intramolecular reaction, thus changing the structure of the apoaequorin into oxygenase. Oxygenase catalyses the oxidation of coelenterazine, and then luminescence occurs with a maximal wavelength of 470 nm and a quantum yield of 0.14, to give a blue fluorescent protein (BFP) and carbon dioxide. Excitation light is not required for the occurrence of luminescence.

As described in O. Shimomura, Tetrahedron Lett. 14(1973)2963-2966 and T. Hirano et al., J. Chem. Soc., Chem. Commun. 2(1994)165-168 (hereinafter, referred to as “reference 1” and “reference 2”, respectively), BFP is composed of apoaequorin bound to coelenteramide, which is oxidized coelenterazine. Luminescence is caused by BPF in an excited state.

However, when resonant energy is transferred from the excited BFP to the chromophores of GFP, the BFP is excited. Green light emerges in returning to the ground state from the excited state. The green light has a maximal wavelength of 510 nm. The green light is the same as light emitted by Aequorea victoria (see F. H. Johnson et al., J. Cell. Comp. Physiol. 60(1962)85-103, J. G. Morin et al., J. Cell. Physiol. 77(1971)313-318, H. Morise et al., Biochemistry. 13(1974)2656-2662, and H. Niwa et al., Proc. Natl. Acad. Sci. USA 93(1996)13617-13622. Hereinafter, referred to as “references 3, 4, 5, and 6”, respectively.).

J. F. Head et al., Nature, 405(2000)372-376 (hereinafter, reference 7) describes the crystal structure of aequorin at 2.3 Å resolution.

Active aequorin is regenerated by allowing apoaequorin to react with coelenterazine, oxygen, and reductant in the absence of calcium ions.

Aequorin is sensitive to very low concentrations of calcium ions. Hence, aequorin can be applied to measure the concentration of calcium ions in cells under physiological conditions. To analyze calcium ions with specificity and high sensitivity utilizing the luminescence of aequorin, highly purified aequorin is required. Therefore, many approaches for purifying aequorin have been studied.

In a known art, apoaequorin is expressed in Escherichia coli. Aequorin is regenerated by adding coelenterazine to apoaequorin in a culture medium and is purified by chromatography. Aequorin has been produced with relatively high purity by such a known art.

However, aequorin purified by such a known art possibly has nonluminescent isoforms. Therefore, measurement of the concentration of calcium ions with aequorin has unstable detection accuracy.

The first problem is that apoaequorin isolated from E. coli according to the known art (see Japanese Unexamined Patent Application Publication No. 1-132397, hereinafter, referred to as “reference 8”) possibly has two isoforms. The reason is that apoaequorin isolated from E. coli is not subjected to gradient elution chromatography. The two isoforms are isolated by gradient elution chromatography and identified as a reduced isoform and an oxidized isoform. From a previous study, the reduced isoform is unsuitable for a quantitative experiment because the N-terminal amino acids of the reduced isoform are possibly lost due to autolysis.

The second problem is that aequorin regenerated from apoaequorin possibly has four isoforms. The reason is that the regenerated aequorin is not subjected to gradient elution chromatography, The four isoforms are isolated by gradient elution chromatography. Some isoforms among the four isoforms cannot fluoresce in the presence of calcium ions. Therefore, measurement of the concentration of calcium ions with aequorin containing the four isoforms has unstable detection accuracy.

### SUMMARY OF THE INVENTION

To solve the above-described problems, it is an object of the present invention to provide a method of purifying aequorin in order to produce highly pure aequorin and a process for measuring the concentration of calcium ions with the highly pure aequorin.

In particular, it is another object of the present invention to quantitatively measure the concentration of calcium ions with high accuracy using highly pure aequorin obtained by purifying aequorin regenerated from apoaequorin expressed in E. coli by DNA recombination techniques.

According to a first aspect of the present invention, there is provided a method of isolating and purifying apoaequorin. The method comprises the steps of isolating and purifying a reduced form and an oxidized form of apoaequorin from crude apoaequorin by chromatography.

According to a second aspect of the present invention, there is provided a method of isolating and purifying aequorin. The method comprises the steps of regenerating aequorin with an oxidized form of apoaequorin, coelenterazine, oxygen, and a reductant, the apoaequorin being produced by the above-method, and isolating and purifying one fluorescent isoform and three nonflurescent isoforms from the regenerated aequorin by chromatography.

According to a third aspect of the present invention, there is provided aequorin isolated by the above-method.

According to a fourth aspect of the present invention, there is provided a method of detecting calcium ions. The method comprised the step of using the aequorin above-mentioned.

According to a fifth aspect of the present invention, there is provided a method of isolating and purifying aequorin. The method comprises the steps of isolating and purifying a fluorescent isoform and a nonfluorescent isoform by chromatography from aequorin regenerated by allowing apoaequorin to react with coelenterazine, oxygen, and a reductant.

In the fifth aspect of the present invention, it is preferred that the fluorescent aequorin has a single isoform and the nonfluorescent aequorin has three isoforms.

According to a sixth aspect of the present invention, there is provided aequorin produced by the above-method.

According to seventh aspect of the present invention, there is provided a method of detecting calcium ions, which comprises the step of using the aequorin above-mentioned.

### BRIEF DESCRIPTION OF THE DRAWINGS

FIG. 1A is a chromatogram of apoaequorin obtained by gradient elution chromatography with a Mono Q HR 10/10 column;

FIG. 1B is the results of native polyacrylamide gel electrophoresis (PAGE) of apoaequorin;

FIG. 2 is a reaction scheme of the luminescence and regeneration of aequorin;

FIG. 3A is a chromatogram of a solution containing regenerated aequorin. The chromatogram is obtained by applying half of the solution containing regenerated aequorin to a column at three hours after mixing and then by performing gradient elution chromatography;

FIG. 3B is a chromatogram of the solution containing regenerated aequorin. The chromatogram is obtained by applying the other half of the solution containing regenerated aequorin to a column at 12 hours after mixing and then by performing gradient elution chromatography; and

FIG. 4 is a graph depicting the dependence of content of aequorin regenerated from recombinant apoaequorin on the elapsed time after mixing.

### DESCRIPTION OF THE PREFERRED EMBODIMENTS

The present invention will now be described in detail.

Apoaequorin produced by E. coli and aequorin regenerated from apoaequorin each have a plurality of isoforms. The inventors have studied a method for isolating these isoforms and found that these isoforms can be isolated and purified by gradient elution chromatography.

In other words, the inventors have developed a technique to quantitatively measure the concentration of calcium ions with high accuracy using highly pure aequorin obtained by regenerating aequorin with apoaequorin expressed in E. coli and then by purifying the resulting aequorin.

That is, the inventors have developed a method for isolating an oxidized isoform and a reduced isoform from apoaequorin obtained by purifying crude apoaequorin by gradient elution chromatography. The crude apoaequorin is produced in E. coli as described in reference 8.

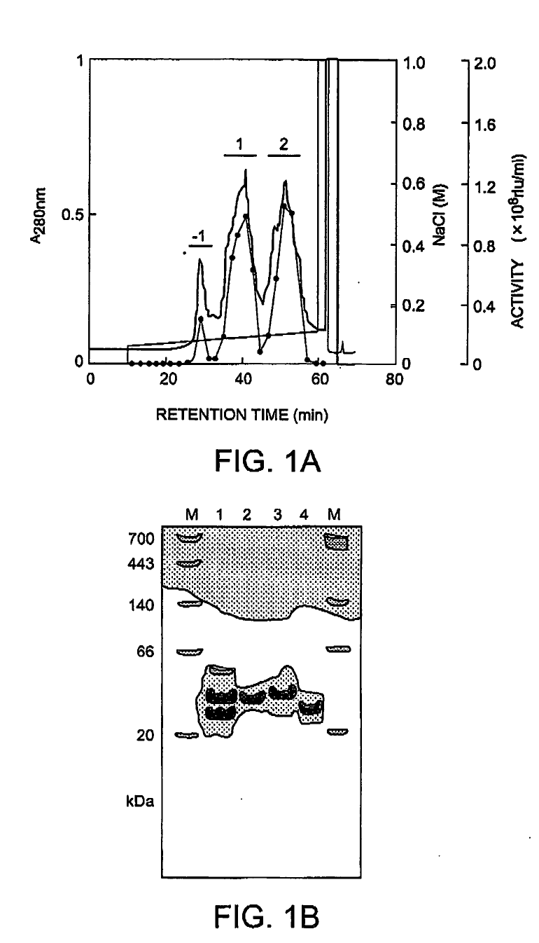

FIG. 1A shows a chromatogram of apoaequorin obtained by gradient elution chromatography with a Mono Q HR 10/10 column. FIG. 1B shows the results of native polyacrylamide gel electrophoresis (PAGE) of apoaequorin. In FIG. 1A, a sample is a peak fraction isolated with a DEAE Sepharose FF column. An equilibration buffer is 30 mM Tris-HCl. An elution buffer is composed of the equilibration buffer further containing 1 M NaCl. In FIG. 1B, molecular weight markers are represented in lane Ms. A peak fraction isolated with a DEAE Sepharose FF column is represented in lane 1. The fractions of peaks −1, 1, and 2 shown in FIG. 1A are represented in lanes 2 to 4, respectively.

In purified recombinant apoaequorin that was stocked for a long period, a sequence of Ala-Asn-Ser-Lys of the N-terminal side is lacking in up to 50% of the fraction of peak 1 (a reduced form of apoaequorin) shown in FIG. 1A, probably because of autolysis caused by peptidase. Hence, a solution containing recombinant apoaequorin possibly has isoforms of apoaequorin depending on a method for purifying or storing the solution. Therefore, a reduced form of apoaequorin is possibly nonuniform.

Consequently, a method for producing highly pure aequorin according to the present invention provides the steps of: regenerating aequorin with an oxidized form of apoaequorin, which is one of the products given by the above-described method; and purifying by gradient elution chromatography.

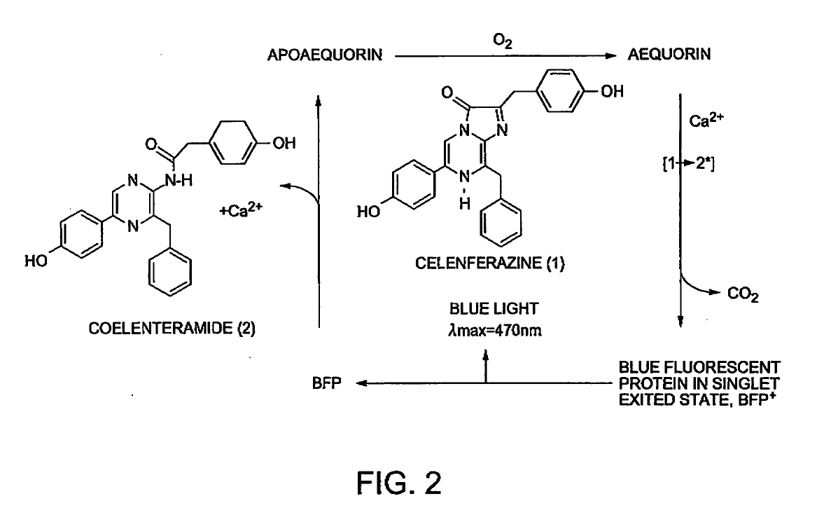

As shown in FIG. 2, aequorin is a complex composed of apoaequorin, which is a protein moiety (apoprotein), coelenterazine (423 Da), which is a substrate, and molecular oxygen (O₂). Binding of calcium ions to aequorin readily causes an intramolecular reaction, thus changing the structure of the apoaequorin into oxygenase. The oxygenase catalyses the oxidation of coelenterazine, and then luminescence occurs with a maximal wavelength of 470 nm and a quantum yield of 0.14, to give a blue fluorescent protein (BFP) and carbon dioxide. BPF is composed of apoaequorin bound to coelenteramide, which is oxidized coelenterazine. Luminescence is caused by BPF in an excited state. After finishing a light emission, BPF is incubated with new coelenterazine, ethylenediaminetetraacetic acid (EDTA), 2-mercaptoethanol, and molecular oxygen. As a result, aequorin can be regenerated.

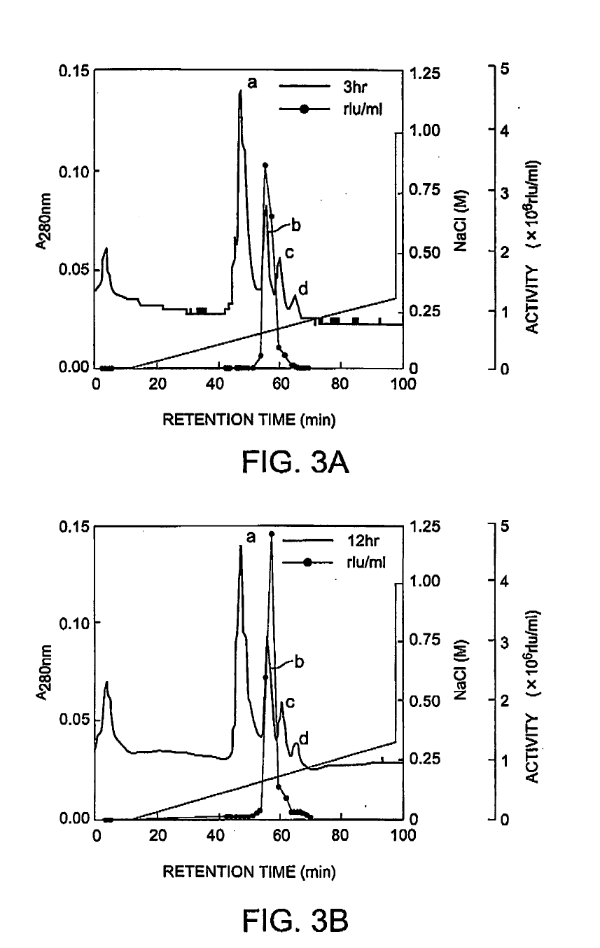

In FIGS. 3A and 3B, a solution containing regenerated aequorin was purified under the following conditions: A solution containing regenerated aequorin was composed of 10 μg of synthetic coelenterazine and 3.4 ml of a solution containing 0.85 mg of apoaequorin. The apoaequorin was obtained from the fraction of peak 2 shown in FIG. 1A. The fraction obtained from peak 2 had been dialyzed with a mixture of 30 mM Tris-HCl (pH 7.6), 10 mM EDTA, and 10 mM 2-mercaptoethanol. Incubation was performed in a cooler. Half of the solution containing regenerated aequorin was applied to the column at three hours after starting incubation and then gradient elution chromatography was performed. FIG. 3A shows the results. The other half of the solution containing regenerated aequorin was applied to the column at 12 hours after starting incubation and then gradient elution chromatography was performed. FIG. 3B shows the results. The column was a Mono Q HR 5/5. An equilibration buffer contained 30 mM Tris-HCl (pH 7.6), 10 mM EDTA, and 10 mM 2-mercaptoethanol. An elution buffer was composed of the equilibration buffer further containing 1 M NaCl. The flow rate was 1 ml/min. Each fraction was 2 ml/tube. As shown in both FIGS. 3A and 3B, aequorin was eluted when the concentration of NaCl was 0.16 M. An assay was performed according to the following procedure: A vial containing 100 μl of sample was placed in a sample chamber of a photometer, and then 1.0 ml of a mixture of 10 mM Tris-HCl (pH 7.6) and 30 mM CaCl₂ was added to the vial. Maximal luminance was represented by relative luminescence units (rlu).

That is, a solution containing aequorin regenerated by mixing an oxidized form of apoaequorin, 10 μg of coelenterazine, EDTA, 2-mercaptoethanol, and molecular oxygen was subjected to gradient elution chromatography. As a result, four peaks a, b, c, and d were observed for both solutions at 3 and 12 hours after mixing as shown in FIGS. 3A and 3B.

The two elution curves just overlapped. Activity was measured by adding an excess of calcium chloride solution to each eluate fraction and then recording the first maximal luminance. Only the fraction of peak b indicated activity. The activity of aequorin after 12 hours was 1.4 times that after three hours, Hence, the fraction obtained from peak b was identified as highly pure aequorin. Peak a represented the amount of apoaequorin remaining in the solution containing regenerated aequorin. The fractions obtained from peaks c and d were apoaequorin or unknown oligomers of aequorin and were formed in the solution during regeneration of aequorin. When the same solution is used in an assay to measure calcium ions, luminance does not accurately indicate an amount of calcium ions because calcium ions are trapped by the luminescent isoform (peak b) and nonluminescent isoforms (peaks a, c, and d). Practical application of aequorin is described in J. R. Blinks et al., Methods in Enzymology 57(1978)292-328 (hereinafter, referred to as “reference 9”).

Embodiments of the present invention will now be described.

(1) Expression and crude purification of recombinant apoaequorin will be described below.

Apoaequorin used in the present invention is a luminescent protein of Aequorea victoria origin. Aequorea victoria is a luminescent creature. The expression and crude purification of recombinant apoaequorin were performed according to a known process (see reference 8).

(2) Purification of apoaequorin will be described below.

In a known art, crudely purified recombinant apoaequorin has been further purified with a DEAE Sepharose FF column to give a fraction showing a single, sharp peak. By dissolving freeze-dried apoaequorin in 20% acetonitrile solution and subjecting the resulting solution to reversed phase high performance liquid chromatography (RP-HPLC), a single, sharp peak has been observed (see S. Inouye et al., Protein Express. Purif. 2(1991)122-126, hereinafter, referred to as “reference 10”). In the present invention, gradient elution chromatography was applied to purify crudely purified recombinant apoaequorin as shown in FIGS. 1A and 1B. As a result, when the concentration of NaCl was 0.08 to 0.1 M, two peaks (peaks 1 and 2) were observed and sometimes a third peak (peak −1) was observed.

In purified recombinant apoaequorin that was stocked for a long period, an N-terminal amino acid sequence was lacking in up to 50% of the fraction of peak 1 (reduced form of apoaequorin) shown in FIG. 1A, probably because of autolysis caused by peptidase. Hence, a solution containing recombinant apoaequorin possibly had isoforms of apoaequorin depending on a method for purifying or storing the solution. Therefore, since apoaequorin in the fraction of peak 1 was possibly nonuniform, the fraction of peak 2 was used for the following purification of aequorin.

As shown in FIG. 1B, apoaequorin purified with a DEAE Sepharose FF column and apoaequorin obtained from the fractions of peaks −1, 1, and 2 were subjected to native-PAGE. As a result, the apoaequorin purified with a DEAE Sepharose FF column showed two apparent bands (lane 1). The fractions obtained from peaks −1 (lane 2) and 1 (lane 3) represented a single band corresponding to the upper band of lane 1, while the fraction obtained from peak 2 (lane 4) represented a single band corresponding to the lower band of lane 1. To each of the fractions was added 2-mercaptoethanol, and then the resulting solutions were subjected to native-PAGE again. As a result, all fractions represented a single band corresponding to the upper band of lane 1. Consequently, the fractions obtained from peaks −1 and 1 represented bands corresponding to the upper bands of lane 1 and were each identified as a reduced form of apoaequorin, while the fraction obtained from peak 2 represented a band corresponding to the lower band of lane 1 and was identified as an oxidized form of apoaequorin.

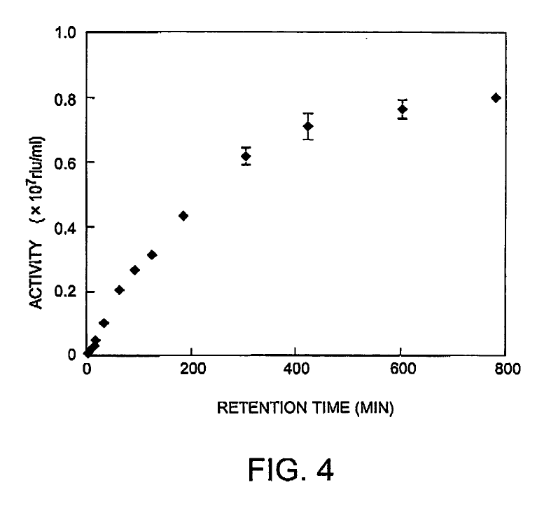

(3) Regeneration of aequorin will be described below referring to FIG. 4.

FIG. 4 is a graph depicting the dependence of content of aequorin regenerated from recombinant apoaequorin on the elapsed time after starting incubation. A solution including regenerated aequorin contained 75 μl of apoaequorin, 15 ml of a mixture of 30 mM Tris-HCl (pH 7.6) and 10 mM EDTA, 60 μl of 2-mercaptoethanol, and 1.5 μg of coelenterazine. Incubation was performed in a cooler. The amount of sample used for an assay was 100 μl. The assay was performed according to the following procedure: A vial containing a sample was placed in a photometer. To the vial was added 1 ml of a mixture of 10 mM Tris-HCl (pH 7.6) and 30 mM CaCl₂. Luminance was measured with one sample at 1, 2, 4, 5, 6, 6.5, and 7 min, two samples at 10 min, and three samples at 15, 30, 60, 90, 120, 180, 300, 420, 600, and 780 min. Maximal luminance was represented by relative luminescence units (rlu).

As shown in FIG. 4, the regeneration of aequorin reached equilibrium after 12 hours. A solution containing regenerated aequorin was composed of oxidized apoaequorin, coelenterazine, EDTA, 2-mercaptoethanol, and molecular oxygen.

(4) Purification of Aequorin will be Described Below.

A solution containing aequorin regenerated as described in item (3) was purified by gradient elution chromatography. Four peaks a, b, c, and d were then observed as shown in FIG. 3A. The luminance of each elution fraction obtained from these four peaks was measured in the presence of calcium ions. As a result, only the fraction obtained form peak b showed activity. Therefore, this fraction obtained from peak b was highly pure aequorin. The fractions obtained from peaks c and d were apoaequorin or unknown oligomers of aequorin and were formed in the solution during the regeneration of aequorin. When the same solution is used in an assay to measure calcium ions, luminance does not accurately indicate the amount of calcium ions because calcium ions are trapped by a luminescent isoform (peak b) and nonluminescent isoforms (peaks a, c, and d).

The present invention will be described in detail based on examples. It is to be understood that the invention is not limited to these examples. The scope of the claims in the present invention is to be interpreted in accordance with the preferred embodiments rather than these examples.

(I) Chemical Agent

EDTA, 2-mercaptoethanol (2.0 ml ampule), and coomassie brilliant blue (CBB) were purchased from Wako Pure Chemical Industries, Ltd. An antifoaming agent CE457 was a gift from NOF CORPORATION. Other chemical agents used were of reagent grade. Coelenterazine was chemically synthesized, and then at least 95% pure coelenterazine was used (see S. Inouye et al, Chem. Lett. (1975)141-144, hereinafter referred to as “reference 11”).

(II) Expression and Crude Purification of Recombinant Apoaequorin.

Apoaequorin was overexpressed by a known method (see reference 11). The expression plasmid used was piP-HE. E. coli WA802 was used as a host. Since the N-terminal of apoaequorin was fused to the E. coli outer membrane protein A (ompA) signal peptide, protein expressed was stored in the periplasm of E. coli to produce into a culture medium. E. coli was cultured in 3.0 L of Luria-Bertani (LB) medium at a temperature of 37° C. with aeration at 3.0 L/min and with moderate shaking. After 20 hours, the culture medium was centrifuged at 5,000×g for 5 min at a temperature of 4° C. The pH of the supernatant was adjusted to 4.2 with 1 M acetic acid (the pl of apoaequorin is 4.7) to precipitate the protein and then was stirred at a temperature of 4° C. for one to two hours. After that, the resulting mixture was centrifuged at 9000×g for 10 min at a temperature of 4° C. The resulting supernatant was decanted, and then the precipitate of crude apoaequorin was dissolved in 30 ml of 1 M TriS-HCl (pH 10). The resulting solution containing crude apoaequorin was dialyzed three times with a mixture of 30 mM Tris-HCl (pH 7.6) and 5 mM CaCl₂ at a temperature of 4° C. Each dialysis was performed for five hours with stirring. The dialysate was centrifuged at 10,000×g for 5 min at a temperature of 4° C. The resulting supernatant was filtered with 0.22 μm filter and stored at 4° C. until the supernatant was subjected to the following purification steps. Apoaequorin dispensed for measuring the activity was cryopreserved at −20° C. until measurement.

(III) Purification of Apoaequorin and Aequorin.

Gradient elution chromatography was performed with a fast protein liquid chromatography (FPLC) system (manufactured by Amersham Biosciences) having a controller LCC-501, a fraction collector FRAC-200, and a two-wavelength ultraviolet monitor installed in a chromatography chamber which was maintained at a temperature of 4° C.

To a column (2.6×30 cm) which was packed with a DEAE Sepharose FF (manufactured by Amersham Biosciences) and then equilibrated with a mixture of 30 mM Tris-HCl (pH 7.6) and 5 mM CaCl₂ was applied 25 ml of a filtered supernatant containing 100 mg of crude apoaequorin. Apoaequorin was eluted with a gradient of 1 M NaCl in a mixture of 30 mM Tris-HCl (pH 7.6) and 5 mM CaCl₂. The flow rate was 4 ml/min, and 12 ml fractions were collected. When the concentration of NaCl was 0.15 to 0.30 M, a fraction of a single, sharp peak assigned to apoaequorin was collected.

A prepacked HiLoad Superdex 26/60 75 pg (2.6×60 cm) column, a Mono Q 10/10 (1.0×10 cm, prepacked with a strong anion-exchange medium) column, and a Mono Q 5/5 (0.5×5.0 cm) column were used for the other purification. All columns were purchased from Amersham Biosciences. Each sample was completely dialyzed at a temperature of 4° C. with an equilibration buffer used for a column and then filtered. To the Mono Q 10/10 column was applied 30 to 40 ml of a fraction containing about 20 mg of apoaequorin isolated with the DEAE Sepharose FF column. To the HiLoad Superdex 26/60 75 pg column were applied 6 ml of each reduced form and oxidized form of apoaequorin isolated with the Mono Q 10/10 column to give a single peak, respectively.

In FIGS. 3A and 3B, a solution containing regenerated aequorin was purified with the Mono Q 5/5 column under the following conditions: The solution containing regenerated aequorin was composed of 10 □g of synthetic coelenterazine and 3.4 ml of a solution containing 0.85 mg of oxidized apoaequorin. The apoaequorin was obtained from the fraction of peak 2 shown in FIG. 1A. The fraction obtained from peak 2 had been dialyzed with a solution containing 30 mM Tris-HCl (pH 7.6), 10 mM EDTA, and 10 mM 2-mercaptoethanol. Incubation was performed in a cooler.

Half of the solution containing regenerated aequorin was applied to the column at three hours after mixing and then gradient elution chromatography was performed. FIG. 3A shows the results.

The other half of the solution containing regenerated aequorin was applied to the column at 12 hours after mixing and then gradient elution chromatography was performed. FIG. 3B shows the results.

The column was a Mono Q HR 5/5. An equilibration buffer contained 30 mM Tris-HCl (pH 7.6), 10 mM EDTA, and 10 mM 2-mercaptoethanol. An elution buffer was composed of the equilibration buffer further containing 1 M NaCl. The flow rate was 1 ml/min. Each fraction was 2 ml/tube. As shown in both FIGS. 3A and 3B, aequorin was eluted when the concentration of NaCl was 0.16 M. An assay was performed according to the following procedure: A vial containing 100 μl of sample was placed in a sample chamber of a photometer, and then 1.0 ml of a solution containing 10 mM Tris-HCl (pH 7.6) and 30 mM CaCl₂ was added to the vial. Maximal luminance was represented by relative luminescence units (rlu). As shown in FIGS. 3A and 3B, four peaks a, b, c, and d were observed for both solutions at 3 and 12 hours after mixing. The two elution curves just overlapped. Activity was measured by adding an excess of calcium chloride solution to each 100 μl of eluate fraction and then recording the first maximal luminance. Only the fraction of peak b indicated activity. The activity of aequorin after 12 hours was 1.4 times that after three hours. Hence, the fraction obtained from peak b was identified as highly pure aequorin. Peak a represented the amount of apoaequorin remaining in the solution containing regenerated aequorin. The fractions obtained from peaks c and d were apoaequorin or unknown oligomers of aequorin and were formed in the solution during regeneration of aequorin. When the same solution is used in an assay to measure calcium ions, luminance does not accurately indicate the amount of calcium ions because calcium ions are trapped by the luminescent isoform (peak b) and nonluminescent isoforms (peaks a, c, and d). Practical application of aequorin is described in reference 8 and J. R. Blinks et al., Mol. Biol. 40(1982)1-114 (hereinafter, referred to as “reference 12”).

(IV) Analysis of Protein.

Collected fractions were analyzed by sodium dodecyl sulfate-polyacrylamide gel electrophoresis (SDS-PAGE) (10% gel) and native-PAGE (gel with a gradient of 5% to 20% and a Tris-Tricine electrophoresis buffer were used) under nonreductive conditions. The gel was stained with coomassie brilliant blue (CBB). The concentration of protein was measured by a Bradford protein assay with bovine serum albumin as the standard. To determine an N-terminal amino acid sequence, bands of protein were transferred to a polyvinylidene fluoride (PVDF) membrane by electroblotting. The sequence was determined with Model 494 Procise Sequencer (manufactured by Applied Biosystems) at University of California, San Diego (UCSD).

(V) Measurement of Activity and Luminescence Intensity of Aequorin.

After apoaequorin was incubated with coelenterazine, EDTA, 2-mercaptoethanol, and molecular oxygen for two hours to regenerate aequorin, the activity of aequorin was measured. A small quantity of a solution containing regenerated aequorin was introduced into a reaction vial, and the vial was placed in a sample chamber of a Hastings-Mitchell photomultiplier-photometer. And then, 1.5 ml of a mixture of 30 mM CaCl and 10 mM Tris-HCl (pH 7.6) was added to the vial. The first maximal luminance was recorded with a chart recorder VP-6712A (manufactured by Matsushita Electric Industrial Co., Ltd.). Relative luminescence units (rlu) were used as the luminance unit.

As described above, the present invention provides a method for quantitatively measuring the concentration of calcium ions with high accuracy using highly pure aequorin obtained by purifying aequorin regenerated from apoaequorin expressed in E. coli by DNA recombination techniques.

Furthermore, a method for isolating and purifying apoaequorin according to the present invention can be applied to the purification of aequorin that is used for the analysis of calcium ions.

1. A method of isolating and purifying apoaequorin, comprising the steps of isolating and purifying a reduced form and an oxidized form of apoaequorin from crude apoaequorin by chromatography.

2. A method of isolating and purifying aequorin, comprising the steps of regenerating aequorin with an oxidized form of apoaequorin, coelenterazine, oxygen, and a reductant, the apoaequorin being produced by the method according to claim 1; and isolating and purifying one fluorescent isoform and three nonflurescent isoforms from the regenerated aequorin by chromatography.

3. Aequorin isolated by the method according to claim 2.

4. A method of detecting calcium ions, comprising the step of using aequorin according to claim 3.

5. A method of isolating and purifying aequorin, comprising the steps of isolating and purifying a fluorescent isoform and a nonfluorescent isoform by chromatography from aequorin regenerated by allowing apoaequorin to react with coelenterazine, oxygen, and a reductant.

6. The method according to claim 5, wherein the fluorescent aequorin has a single isoform and the nonfluorescent aequorin has three isoforms.

7. Aequorin produced by the method according to claim 6.

8. A method of detecting calcium ions, comprising the step of using aequorin according to claim 7.
